(19) United States
(12) Patent Application Publication
(10) Pub. No.: US 2005/0202050 A1
(43) Pub. Date: Sep. 15, 2005

(54) Single tank process for preparing tannate liquid and semi-solid dosage forms

(76) Inventors: Jeffrey S. Kiel, Gainesville, GA (US); H. Greg Thomas, Villa Rica, GA (US)

Correspondence Address: KING & SCHICKLI, PLLC, 247 NORTH BROADWAY, LEXINGTON, KY, 40507, US

(21) Appl. No.: 10/799,302

(22) Filed: Mar. 12, 2004

(51) Int. Cl. A61K 31/7024; A61K 9/00

(52) U.S. Cl. 424/400; 514/23

(57) ABSTRACT

A manufacturing process for tannate salt complexes of pharmaceutically active compounds includes the steps of dissolving a salt or free base of an active pharmaceutical ingredient in a pharmaceutically acceptable liquid in the presence of a dispersing agent and tannic acid to form a dispersion and combining the tannate salt complex of the active pharmaceutical ingredient without isolation or purification with pharmaceutically acceptable excipients to generate a therapeutic dosage form.

### BACKGROUND OF THE INVENTION

1. Field of the Invention

The present invention relates generally to the field of tannate chemistry and more specifically to methods for processing tannate pharmaceutical suspensions.

2. Description of the Prior Art

The use of tannate suspensions in pharmaceutical products is well-known. U.S. Pat. No. 6,287,597 describes tannate suspensions containing pyrilamine tannate and phenylephrine tannate. The suspension is prepared in a conventional manner such that one teaspoon contains 30 mg pyrilamine tannate and 5 mg phenylephrine tannate with benzoic acid, coloring agent, natural and artificial flavors, glycerin, kaolin, magnesium aluminum silicate, methyl paraben, pectin, purified water, saccharin, sodium hydroxide, and sucrose or sorbitol.

The January 1990 issue of Annals of Allergy, Volume 64, describes combinations of chlorpheniramine tannate, pyrilamine tannate and phenylephrine tannate. An article in Clinical Medicine, dated September 1965, pages 1475-1478, describes tablets of pyrilamine tannate, chlorpheniramine tannate, and amphetamine tannate. Phenylephrine tannate compositions are disclosed in U.S. Pat. No. 5,599,846 and phenylephrine tannate and chlorpheniramine tannate compositions are disclosed in U.S. Pat. No. 6,037,358.

None of these references suggest or describe the production of a suspension by means of an in-situ conversion of the active ingredient to the tannate salt using the method described herein to provide a dosage form which affords a sustained release of the active ingredient over prolonged intervals of time. Since the prolonged drug release character of the tannate salt enables the development of less frequent dosing regimens, such a suspension is needed to improve patient compliance with dosage requirements.

In addition, none of these references describe a solution to the inherent difficulties encountered in preparing tannate pharmaceutical products. Because of the size of the tannic acid molecule, the percentage of active free-base within the tannate salt complex is significantly lower than that in other salt forms such as the hydrochloride or maleate. Further the variable purity of the commercially available tannate salts leads to variation in the stoichiometry of the active free-base to tannic acid in the tannate salts from batch to batch. The low percentage of active free-base in the tannate salt and the variable stoichiometry in the commercially available tannate salts act synergistically to increase product variability.

This problem was noted in U.S. Pat. Nos. 5,599,846 and 5,663,415, and causes significant processing challenges during manufacture and increases the likelihood that commercially available pharmaceutical products contain variable and in some instances, sub-therapeutic levels of the active drug substances. Therefore, it would be desirable if liquid and semi-solid dosage form pharmaceutical compositions containing tannate salts of active ingredients could be prepared with reduced variability in active drug content and increased certainty that the active drug is delivered within the therapeutic range. The present invention provides an economical solution to the preceding difficulties.

### SUMMARY OF THE INVENTION

The present invention provides a manufacturing process for production of tannate salt complexes of pharmaceutically active compounds and subsequent incorporation thereof, into a therapeutic liquid or semi-solid dosage form in an efficient and cost effective manner. By starting with a commonly available salt or free base of the active pharmaceutical ingredient, which is subsequently converted and incorporated in-situ as a tannate salt complex, removing the necessity of an additional isolation step, the invention provides a reproducible method to manufacture liquid or semi-solid products containing tannate salt complexes as active ingredients with decreased variability in dose.

In particular, the invention provides a means for reducing dose variability in pharmaceutical products containing tannate salts as active ingredients. The invention also may afford a prolonged release of active pharmaceutical ingredients, thereby facilitating a reduction in the frequency of drug administration and improving patient compliance. In addition, tannate salts have been found to have better organoleptic properties such as taste in comparison to other salts or freebase forms.

### DETAILED DESCRIPTION OF THE INVENTION

The present invention provides a novel manufacturing process for the conversion of one or more active pharmaceutical ingredients (“API”) into tannate salt complexes while directly incorporating the complexes into a therapeutic liquid or semi-solid dosage form which also may include non-tannate API's. The first step of this process is to create a tannic acid dispersion by combining tannic acid and a suspending agent in water or other pharmaceutically acceptable liquid. Other pharmaceutically acceptable ingredients may also be added to the dispersion, including but not limited to thickeners or buffering agents. The presence of the suspending agent prevents the aggregation of the tannate salt complex formed and promotes uniformity in the suspension. After the tannic acid is thoroughly dispersed, a commonly available salt of a pharmaceutical active ingredient is added to the mixture and is dissolved. Upon dissolving, the tannate salt spontaneously forms. Preparing a tannic acid dispersion and adding a commonly available salt or free base form of a pharmaceutical active ingredient in this manner is sufficient to facilitate the conversion of the active ingredient to the tannate salt form. The resulting suspension is processed directly into liquid or semi-solid-dosage pharmaceutical products as necessary.

Naturally occurring tannic acid comprises a mixture of compounds. They are considered to be secondary metabolites, with a molecular weight of 500-5000 Da, that have no specific metabolic function. They are complex phenol-rich polymers found in many foods. As with many natural polymers, a rigorous chemical definition of tannins is difficult. In general two classes are distinguished—the hydrolyzable and the condensed tannins. Hydrolyzable tannins or tannic acids are referenced in the various pharmacopeias and are composed of gallic acid or its condensation product ellagic acid esterified to the hydroxyl groups of glucose.

Hydrolyzable tannins are molecules with a polyol (generally D-glucose) as a central core, with the hydroxyl groups of the carbohydrate partially or totally esterified with phenolic groups. They derive their name from their propensity to be hydrolyzed by mild acids or mild bases to yield carbohydrates and phenolic acids. Synthetic tannic acid may comprise a purified form of any of the components of naturally occurring tannic acid.

The present invention may utilize tannic acid of either a natural or synthetic source. The term “tannic acid” herein refers to either natural or synthetic tannic acid as described above.

Tannate salts have been found to have better organoleptic properties such as taste in comparison to other salts or free base forms. In comparison to typical salt forms, the tannate salt of the active pharmaceutical ingredient (API) is a significantly larger molecule that is typically less soluble, which affords absorption of the API over prolonged intervals of time, reducing the frequency of administration and thereby improving patient compliance.

Traditionally, a tannate salt is prepared by reacting the free base of the API with tannic acid in the presence of a volatile solvent, usually isopropanol or water, for designated times and temperatures. After the completion of the reaction, the mixture is filtered, washed and vacuum dried to obtain the tannate salt. The conditions required by the prior art for the isolation of the tannate salt often lead to decreased yield and purity. The yield of the products using such methods varies from about 70% when using the isopropanol route to 90-97% using the water method. The purity of the tannate salt produced as described above is variable. The purity ranges from 85-90% using the isopropanol route to about 90-98% using the water route.

The present invention provides an efficient means for improving the yield of the tannate salt and decreasing variability of dose in the final product. The conversion process requires the presence of basic functional groups such as amines in the molecular structure of the API. The source of tannic acid is natural or synthetic. The formation of the tannate salt is by reaction of amine groups (in the 1°, 2°, 3°, 4°, or amphoteric configuration) or of the other basic functional groups with tannic acid. The amount and ratio of dispersing agent and tannic acid required for the completion of the reaction is determined by the molecular configuration and concentration of the API.

The API tannate salt complex or complexes obtained from the conversion process may then be directly processed into a pharmaceutical product. Other pharmaceutically acceptable excipients such as thickener, sweetening, coloring, flavoring, preservative, suspending, and stabilizing agents may be added as necessary.

By starting with a known amount of commonly available salt or the free base form of the API which is subsequently converted and incorporated as a tannate salt complex into a liquid or semi-solid dosage form without additional purification, the invention provides an efficient and reproducible method to manufacture products containing tannate salt complexes as active ingredients. Since the tannate salt complex of the API is generated and incorporated into the dosage form during the manufacturing process, the need to isolate the tannate salt is eliminated and, perhaps most significantly, the stoichiometry of the tannate salt is uniform from batch to batch resulting in decreased dosage variability.

The following is a non-exclusive list of active pharmaceutical ingredients that may be used in this process:
1. carbinoxamine
2. chlorpheniramine
3. dexchlorpheniramine
4. phenazopyridine
5. pyrilamine
6. pheniramine
7. diphenhydramine
8. bromdiphenhydramine
9. triplennamine
10. brompheniramine
11. loratadine
12. desloratidine
13. fexofenadine
14. carbetapentane
15. dextromethorphan
16. phenylephrine
17. pseudoephedrine
18. ephedrine
19. oxycodone
20. morphine
21. physostigmine
22. cimetidine
23. amantidine
24. fluvoxamine
25. sertraline
26. chlorpromazine
27. imipramine
28. amitryptyline
29. prochlorperazine
30. cetirizine
31. hydroxyzine
32. promethazine
33. acrivastine
34. triprolidine
35. meclizine
36. dimenhydrinate
37. doxylamine
38. diphenylpyrilamine
39. trimeprazine
40. chlorcylizine
41. triphennamine
42. codeine
43. cyproheptadine
44. phenyltoloxamine
45. clemastine
46. famotidine
47. hydrocodone
48. methscopolamine
49. neostigmine
50. gabapentin
51. lithium compounds
52. dopamine
53. bromocriptine
54. carbamazepine
55. desipramine
56. nortriptyline
57. quinidine
58. procainamide
59. ranitidine
60. quinine

The excipients commonly used in the formulations are as follows: magnesium aluminum silicate (MAS) and xanthan gum as anti-clumping agents; polyvinyl pyrrolidone (PVP), hydroxypropylmethylcellulose (HPMC E-10), and glycerin as thickeners; sucrose, saccharin sodium, magnasweet MM-100, and sucralose, as sweetening agents; sodium citrate and sodium phosphate as a buffering agents; methylparaben, propylparaben, and sodium benzoate as preservatives; and grape, cotton candy, artificial strawberry-banana, and artificial strawberry flavor as flavoring agents. Of course, this list should be considered as illustrative in nature and not as restrictive. Thus, other unnamed excipients could be utilized. Active ingredients not present as tannate salt complexes also can be included in the formulation.

The pharmaceutically acceptable liquid used to suspend the active ingredient salt or free base and tannic acid is preferably water. However, other pharmaceutically acceptable liquids can be substituted for water such as isopropyl alcohol, ethanol, propylene glycol, mineral oil or mixtures thereof. The addition of the pharmaceutically acceptable liquid leads to the dissociation of the active ingredient salt into its free-base and conjugate acid forms which facilitates the formation of the tannate salt complex of the active ingredient.

The following examples illustrate the conversion process and subsequent incorporation of the tannate salt complexes into suitable liquid or semi-solid dosage forms. The following examples are only intended to illustrate the present invention, and are not intended to limit its scope. Many other pharmaceutical active ingredients may be converted to the tannate salt form in like manner, and various equivalents to the ingredients may be substituted without departing from the spirit and scope of the present invention.

### EXAMPLE 1

Preparation of Pyrilamine Tannate, Phenylephrine Tannate, and Dextromethorphan Tannate Suspension

A typical formulation for a suspension containing tannate salts active ingredients is detailed in the table below.

| Raw Material | % w/v | Target Wt. (Kg) | |
|---|---|---|---|
| Pyrilamine Maleate | 0.320% | *5.604 | |
| Phenylephrine HCI | 0.100% | *1.751 | |
| Dextromethorphan HBr | 0.300% | *5.253 | |
| Tannic Acid | 0.801% | *14.026 | |
| Sucrose | 10.000% | 170.000 | |
| Glycerin | 7.500% | 127.500 | |
| Magnesium Aluminum | 0.800% | | |
| Silicate | | 13.600 | |
| Xanthan Gum | 0.450% | 7.650 | |
| Sodium Citrate Dihydrate | 1.000% | 17.000 | |
| Citric Acid | 0.400% | 6.800 | |
| Methylparaben | 0.200% | 3.400 | |
| Magnasweet MM-100 | 0.300% | 5.100 | |
| Sodium Benzoate | 0.100% | 1.700 | |
| Sucralose | 0.200% | 3.400 | |
| FD&C Blue #1 | 0.004% | 0.068 | |
| FD&C Red #40 | 0.015% | 0.255 | |
| Artificial Grape Flavor | 1.300% | 22.100 | |
| Purified Water | qs to | 1374.793 | |
| | weight | | |
| Total: | 100.00% | 1780.00 | Kg |

*Includes overage of 3% for Pyrilamine, Phenylephrine, Dextromethorphan and Tannic Acid.

In separate vessels, the FD&C Red No. 40 and FD&C Blue No. 1 are dissolved in 1 L purified water.

1200 Kg of water are added to a third vessel. Then, to the 1200 Kg, the dextromethorphan HBr is added. Pressure is maintained at or below 20 mm Hg during mixing. The Pyrilamine Maleate is then added. Again, mixing is continued for approximately 20 minutes at or below 20 mm Hg. The phenylephrine HCl is then added. Again, mixing is continued for approximately 20 minutes at or below 20 mm Hg. At this point, the Sucrose, Sucralose, Magnsweet MM-100, and Artificial Grape Flavor are added. Mixing is continued for approximately 30 minutes at or below 20 mm Hg. The glycerin, methylparaben, and Sodium Benzoate, are then added. Mixing is continued for approximately 30 minutes at or below 20 mm Hg. The vacuum is removed, and mixing is continued for an additional 5 minutes. Finally, the pH of the formulation is adjusted if necessary to be within the range of 4.8-5.2.

### EXAMPLE 2

Preparation of Dexchlorpheniramine Tannate and Pseudoephedrine Tannate Suspension

| Raw Material | % w/v | Target Wt. (kg) | |
|---|---|---|---|
| Dexchlorpheniramine Maleate | 0.032% | 0.048 | |
| Pseudoephedrine HCI | 0.600% | 0.912 | |
| Tannic Acid | 1.037% | 1.576 | |
| Sucrose | 10.000% | 15.200 | |
| Sodium Saccharin | 0.500% | 0.760 | |
| Glycerin | 7.500% | 11.400 | |
| Magnesium Aluminum | 0.800% | | |
| Silicate | | 1.216 | |
| Xanthan Gum | 0.450% | 0.684 | |
| Sodium Citrate Dihydrate | 1.000% | 1.520 | |
| Citric Acid | 0.200% | 0.304 | |
| Methylparaben | 0.200% | 0.304 | |
| Sodium Benzoate | 0.100% | 0.152 | |
| FD&C Red #40 | 0.010% | 0.015 | |
| Artificial Strawberry-banana | 0.800% | | |
| Flavor | | 1.216 | |
| Purified Water | qs to | N/A | |
| | volume | | |
| Total: | 100.00% | 40.000 | gallons |

The formulation is prepared as follows. One hundred-ten Kg of purified water is added to a separate vessel. Then, the sodium citrate dihydrate and citric acid are added to the purified water and are mixed until they are dissolved completely. The MAS and xanthan gum are then added and thoroughly dispersed. After this, the tannic acid is added and thoroughly dispersed. At this point, the dexchlorpheniramine Maleate is added. Mixing is continued for approximately 20 minutes. The pseudoephedrine is then added and mixing is continued again for approximately 20 minutes. The sucrose, and saccharin sodium are then added and are dispersed. The artificial strawberry-banana and FD&C Red #40 are added and blended for approximately 5 minutes. After adding and dispersing the glycerin, methylparaben, and sodium benzoate, the pH of the formulation is adjusted to within the range of 4.8-5.2.

In summary, the present invention relates to an efficient and cost effective process for the production of tannate salt complexes of pharmaceutically active compounds and subsequent incorporation thereof into a therapeutic liquid or semi-solid dosage form. Advantageously, the present process involves fewer steps than known prior art production methods. As a consequence, there is less material and equipment handling and therefore, less potential for introducing contamination into the product or experiencing human error in production. Further, greater productivity is achieved.

As an additional benefit, the present process may be completed in a single tank or vessel. Consequently, less equipment is used than in prior art processes and cleanup time and expense after processing is significantly reduced. This further increases overall production efficiency.

1. A manufacturing process for the conversion and incorporation of a salt or free base of an active pharmaceutical ingredient into a therapeutic liquid or semi-solid dosage form, the process comprising the steps of:
(a) dissolving the salt or free base of the active pharmaceutical ingredient in a pharmaceutically acceptable liquid in the presence of a dispersing agent and tannic acid under stirring, to form a dispersion wherein the tannic acid component is of either a natural or synthetic source;
(b) combining the tannate salt complex of the active pharmaceutical ingredient without isolation or purification with pharmaceutically acceptable excipients to generate a therapeutic dosage form.

2. The process according to claim 1 wherein the dispersing agent provided in step (b) is selected from the group consisting of magnesium aluminum silicate, xanthan gum and cellulose compounds.

3. The process according to claim 1 wherein the pharmaceutically acceptable liquid in step (a) is selected from the groups consisting of purified water, isopropyl alcohol, ethanol, glycerin, propylene glycol, mineral oil and mixtures thereof.

4. The process according to claim 1 wherein without isolation or purification of the tannate salt or complex of the active pharmaceutical ingredient, the additional steps are:
(c) separately adding one or more of the following; thickening, suspending, coloring, sweetening and flavoring agents to water under stirring, to form a dispersion;
(d) adding the tannate salt suspension from step (a) to the dispersion in step (c), under stirring to form a mixture containing the tannate salt complex of the active pharmaceutical ingredient;
(e) separately adding one or more of the following; preservative, pH adjusting and anti-caking agents to a pharmaceutically acceptable liquid under stirring to form a dispersion; and
(f) adding the dispersion from step (e) to the mixture from step (d) under stirring, to generate a suspension dosage form, at a pH range of 3.5-8.0.

5. A manufacturing process for the conversion and incorporation of a salt or free base of an active pharmaceutical ingredient selected from the group consisting of an antihistamine, a decongestant, an antitussive and an anticholinergic for incorporation into a therapeutic liquid or semi-solid dosage form, the process comprising the steps of:
(a) dissolving the salt or free base of the active pharmaceutical ingredient in a pharmaceutically acceptable liquid in the presence of a dispersing agent and tannic acid under stirring, to form a dispersion wherein the tannic acid component is of either a natural or synthetic source;
(b) combining the tannate salt complex of the active pharmaceutical ingredient without isolation or purification with pharmaceutically acceptable excipients to generate a therapeutic dosage form.

6. The process according to claim 5 wherein the antihistamine active pharmaceutical ingredient is selected from the group consisting of: carbinoxamine, chlorpheniramine, pyrilamine, pheniramine, phenindamine, diphenhydramine, bromodiphenhydramine, brompheniramine, loratadine, desloratadine, fexofenadine, cetirizine, hydroxyzine, promethazine, acrivastine, triprolidine, meclizine, dimenhydrinate, triplennamine, doxylamine, diphenylpyrilamine, trimeprazine; and chlorcylizine.

7. The process according to claim 5 wherein the antitussive active pharmaceutical ingredient is selected from the group consisting of: carbetapentane, dextromethorphan, diphenhydramine, codeine, hydrocodone, oxycodone, and morphine.

8. The process according to claim 5 wherein the decongestant active pharmaceutical ingredient is selected from the group consisting of: phenylephrine, pseudoephedrine, ephedrine, diphenhydramine, cyproheptadine, phenyltoloxamine, and clemastine.

9. The process according to claim 5 wherein the anticholinergic active pharmaceutical ingredient is methscopolamine.

10. The process according to claim 5 wherein the antihistamine and decongestants active ingredients are provided as the bitartrate, maleate, citrate, chloride, bromide, acetate or sulfate salt.

11. The process according to claim 5 wherein the tannic acid provided in step (a) is natural or synthetic.

12. The process according to claim 5 wherein a mixture of antihistamine tannate and decongestant tannate salts are formed in step (b).

13. The process according to claim 12 wherein the antihistamine tannate and decongestant tannate salts in step (b) comprise carbetapentane tannate, phenylephrine tannate and pyrilamine tannate.

14. The process according to claim 12 wherein the antihistamine tannate and decongestant tannate salts in step (b) comprise pyrilamine tannate and phenylephrine tannate.

15. The process according to claim 12 wherein the antihistamine tannate and decongestant tannate salts in step (b) comprise pseudoephedrine tannate and chlorpheniramine tannate.

16. A manufacturing process for the conversion and incorporation of a salt or free base of an active pharmaceutical ingredient into a therapeutic liquid or semi-solid dosage form, the process comprising the steps of:
dissolving the salt or free base of the pharmaceutical ingredient and tannic acid in a pharmaceutically acceptable liquid to form a dispersion; and
adding at least one pharmaceutically acceptable excipient to said dispersion to generate a therapeutic dosage form.

17. The process of claim 16, further including completing the dissolving and adding steps in a single vessel.

18. The process of claim 17, further including generating said therapeutic dosage form without isolating or purifying a resulting tannate salt complex of the active pharmaceutical ingredient.

19. The process of claim 16, further including generating said therapeutic dosage form without isolating or purifying a resulting tannate salt complex of the active pharmaceutical ingredient.
